(19) United States
(12) Patent Application Publication
(10) Pub. No.: US 2023/0364270 A1
(43) Pub. Date: Nov. 16, 2023

(54) LIPOSOME-ASSISTED IMAGING OF VASCULAR INFLAMMATION

(71) Applicants: Behnam REZAI JAHROMI, Helsinki (FI); Vladimir ZAMOTIN, Helsinki (FI); Riikka TULAMO, Helsinki (FI); Eliisa NETTI, Helsinki (FI); Aki LAAKSO, Helsinki (FI); Christian CODE, Helsinki (FI); Mika NIEMELÄ, Helsinki (FI); HELSINGIN JA UUDENMAAN SAIRAANHOITOPIIRIN KUNTAYHTYMÄ, HUS (FI)

(72) Inventors: Behnam REZAI JAHROMI, Helsinki (FI); Vladimir ZAMOTIN, Helsinki (FI); Riikka TULAMO, Helsinki (FI); Eliisa NETTI, Helsinki (FI); Aki LAAKSO, Helsinki (FI); Christian CODE, Helsinki (FI); Mika NIEMELÄ, Helsinki (FI)

(21) Appl. No.: 18/026,020

(22) PCT Filed: Sep. 9, 2021
(86) PCT No.: PCT/FI2021/050601

§ 371 Date: Mar. 13, 2023

(30) Foreign Application Priority Data
Sep. 11, 2020 (WO) PCT/FI2020/050583

(51) Int. Cl. A61K 49/00; A61K 47/69; A61K 47/68; A61K 47/54

(52) U.S. Cl. CPC A61K 49/0084; A61K 49/0058; A61K 49/0043; A61K 47/6913; A61K 47/6871; A61K 47/6843; A61K 47/545

(57) ABSTRACT

Described herein are liposomes that can be capable of targeting within blood vessels to an intended tissue area presenting at least one vascular inflammatory marker and enhancing imaging contrast therein. Described herein are aspects of a targeting liposome that can carry antibodies against at least one vascular inflammatory marker and a contrast agent to an intended tissue area presenting the at least one vascular inflammatory marker whereby the liposomes can be capable of anchoring to the intended vascular inflammation site and enhancing imaging contrast of it. Also described herein are methods of using the targeting liposomes for anchoring the liposomes to vascular inflammation and imaging vascular inflammation.

### TECHNICAL FIELD

The present disclosure generally relates to compositions and methods for imaging vascular inflammation. In particularly, however not exclusively, the present disclosure concerns targeting liposomes for targeting imaging agents to vascular inflammation and related methods for imaging vascular inflammation.

### BACKGROUND

There exists a variety of compositions of imaging agents which provide intravenous contrast medium enhancements for imaging and detecting vascular inflammations. However, the imaging contrast enhancement provided by these imaging agents are dependent on many complex factors, including for example the type of media, volume, concentration, imaging technique, and tissue characteristics. These factors and diffusion of imaging agents outside the vascular space severely limit imaging of inflammation site by degrading lesion conspicuity and imaging quality. With many of these agents, and with most of the commonly used contrast agents, the enhancement cannot be directed against a specific target such as protein in tissue. Especially in the imaging and detecting of intracranial aneurysms (IA) the present imaging agents provide insufficient image to distinguish inflammatory cells or other inflammation associated markers (i.e. inflammatory markers) from healthy cells and tissues. As such, there exists a need for improved compositions and methods which enhance imaging and detecting of vascular inflammation and, for example, provide such a lesion conspicuity and imaging quality of imaging IAs and other vascular pathologies which allow a way of detailed detection and analysis of aneurysm or vascular wall. Accordingly, improved imaging methods and imaging agents will have broad clinical utility.

### SUMMARY

An object of the invention is to present a composition and method for intravenous contrast medium enhancement for imaging and detecting vascular inflammation so that at least deficiencies related to prior art can be reduced. The objects of the invention are obtained with a targeting liposome which carries agents for anchoring and enhancing imaging contrast to vascular inflammation and related methods, which are characterized in what is presented in the independent claims. Some advantageous embodiments of the invention are presented in the dependent claims.

Described herein are aspects of a targeting liposome (i.e. immunoliposome) for use in the imaging of aneurysm (in vivo), the targeting liposome comprises antibodies against at least one vascular inflammatory marker associated with aneurysm and a label and/or a contrast agent.

Also described herein are aspects of a targeting method for anchoring a targeting liposome which carries antibodies against or for at least one vascular inflammatory marker and a label and/or a contrast agent to an intended tissue area secreting the at least one vascular inflammatory marker associated with aneurysm, the method comprises administering the liposome to a subject.

Also described herein are aspects of an imaging method for imaging vascular inflammation, the imaging method comprises detecting the intended tissue area by using an imaging method that detects the label and/or the contrast agent carried in the targeting liposome.

Also described herein are aspects of a targeting liposome. The targeting liposome can be used in the targeting method and the imaging method disclosed in this document. The targeting liposome comprises at least one type of lipid and an antibody against at least one inflammatory marker associated with aneurysm. The liposome further comprises at least one label and/or a contrast agent.

Also described herein are aspects of a vehicle for use in delivering active pharmaceutical ingredient (API), wherein the targeting liposome disclosed in this document is utilised for delivering the API onto the intended tissue area presenting or secreting the at least one vascular inflammatory marker associated with aneurysm.

Also described herein are aspects of a carrier for use in delivering label and/or contrast agent, wherein the targeting liposome disclosed in this document is utilised for delivering the label and/or contrast agent to the intended tissue area secreting the at least one vascular inflammatory marker associated with aneurysm.

An advantage of the invention is that it may allow an anchoring of the targeting liposome at the vascular inflammatory tissue site, for example at the site of aneurysm that is intended to be imaged. It may further extend the imaging window for obtaining acceptable contrast.

An advantage of the invention is further that it may allow carrying the API to the intended tissue area (i.e. target) or into close proximity of the target at the site of vascular inflammation.

An advantage of the invention is further that it may provide a safe, non-invasive way of imaging vascular inflammation and states, disorders, or diseases that are caused by vascular inflammation.

Still other aspects, embodiments, and advantages of these example aspects and embodiments are discussed in detail below. Moreover, it is to be understood that both the foregoing information and the following detailed description are merely illustrative examples of various aspects and embodiments. Exemplifying and non-limiting embodiments are mutually freely combinable unless otherwise explicitly stated.

The embodiments in the following detailed description are given as examples only and someone skilled in the art can carry out the basic idea of the invention also in some other way than what is described in the description. Most embodiments can be actualised in a variety of combinations with other embodiments. Though the description may refer to a certain embodiment or embodiments in several places, this does not imply that the reference is directed towards only one described embodiment or that the described characteristic is usable only in one described embodiment. The individual characteristics of a plurality of embodiments may be combined and new embodiments of the invention may thus be provided.

Furthermore, the presented considerations concerning the various embodiments of the targeting liposome may be flexibly applied to the embodiments of the methods mutatis mutandis, and vice versa, as being appreciated by a skilled person.

### BRIEF DESCRIPTION OF THE DRAWINGS

FIG. 1 shows schematically an example of anchoring of the targeting liposome with a vascular inflammatory marker.

FIG. 2 shows a scheme of the main steps of synthesis of the targeting liposome according to an embodiment.

FIG. 3 shows measured fluorescence spectra of the targeting liposomes with encapsulated carboxyfluorescein according to an embodiment.

FIG. 4 shows dynamic light scattering (DLS) spectra according to embodiments.

FIGS. 5a-c, 6a-c and 7a-c show fluorescence stainings for unconjugated, and conjugated targeting liposomes according to embodiments.

### DETAILED DESCRIPTION

The applicant has found that imaging contrast and quality of vascular inflammation by utilising the clinically suitable imaging method, such as in magnetic resonance imaging (MRI), can be enhanced and targeted to vascular inflammation by encapsulating imaging contrast agent and/or imaging label inside specific targeting liposomes carrying on its surface specific antibodies against vascular inflammatory marker presented at the vascular inflammation. The targeting liposome according to the present disclosure may allow to increase the contrast agent concentration and prolong the contrast agent presence at the inflammatory site compared to the present contrast agents and methods whereby quality and contrast of imaging of inflammatory site may be improved. By attaching the targeting liposomes to the expressed or overexpressed biological markers in an aneurysm wall or vascular wall in other vascular pathologies, a reliable diagnostic tool for objective evaluation of vascular pathologies, and for example of rupture risk of the aneurysm, can be attained. The targeting liposomes and methods provided herein can also be utilized to identify other states, disorders or diseases related to vascular inflammation, such as atherosclerosis, a stroke, an abscess, an infarct, an ischemia and/or a vasculitis, for example.

In the present disclosure “a label and/or contrast agent” stands for a label and/or contrast agent that can be visualized by an imaging method.

In the present disclosure “a magnetic or luminescent or fluorescent moiety” stands for a (organic) substructure of the molecule with magnetic, luminescent or fluorescent properties, respectively.

In the present disclosure “a lipid capable of self-assembling into an amphiphilic colloidal form of the liposome” stands for a type of lipid that favors liposome-formation, preferably based on its electrochemical properties.

In the present disclosure “a polymeric excipient capable of stabilizing the amphiphilic colloidal form of the liposome” stands for a polymer that stabilizes the structure of the liposome.

In the present disclosure “an active pharmaceutical ingredient” or “API capable of producing an intended effect on an inflammation” stands for an active pharmaceutical ingredient (API) that has a therapeutic anti-inflammatory function.

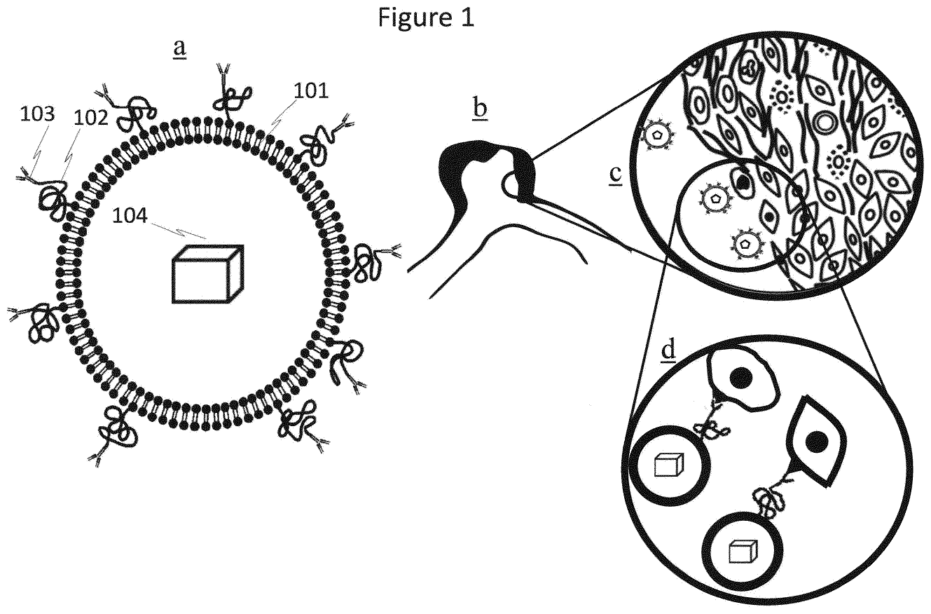

FIG. 1 shows schematically an example of anchoring of the targeting liposome with a vascular inflammatory marker. Panel (a) shows a targeting liposome, wherein 101 is a lipid core; 102 is polyethylene glycol (PEG) chain attached to the lipid core; 103 is an antibody attached to the outer surface of the targeting liposome via the PEG chain; and 104 is a label and/or contrast agent enclosed inside the targeting liposome. Panel (b) shows an aneurysm dome. Panel (c) shows an aneurysm wall with mural and inflammatory cells. Panel (d) shows binding (i.e. anchoring) between the antibody of the targeting liposome and the epitope (a.k.a. antigenic determinant) of the vascular inflammatory marker.

The present disclosure provides compositions and methods for imaging and detecting a specific target in the vasculature of a subject, for example for detecting and evaluating vascular inflammation. The imaging of vascular inflammation is important, for example, for the prediction and/or diagnosis of localized and generalized diseases and disorders and/or organ, tissue, or vessels damage (e.g., ischemic, inflammated, infected, and the like).

The vascular imaging (e.g., imaging of specific vascular sites), can be performed using routine imaging method known in the art. For example, the imaging method is selected from the group consisting of magnetic resonance imaging (MRI), magnetic resonance angiography (MRA), x-ray imaging, computed tomography (CT), computed tomography angiography (CTA), positron emission tomography (PET), single-photon emission computed tomography (SPECT), and Digital subtraction angiography (DSA).

Aneurysms can be divided into different types based on their shape and structure. Saccular aneurysms are the most common type of intracranial aneurysm (IA) and responsible for 70% of all subarachnoid haemorrhage (SAH) cases while in 20% of the cases the origin cannot be identified and where the rest are caused by ruptured arteriovenous malformations (AVMS) and fusiform aneurysms. The morphology of aneurysm wall is different from healthy arterial wall which difference may be detectable by utilizing the targeting liposomes and methods disclosed in this document. Development of aneurysms is a complex process that consists of endothelial erosion, thrombosis in lumen, atherosclerotic chances, inflammation, death e.g. apoptosis of smooth muscle cells and reorganization of extracellular matrix. IAs most often form in bifurcation sites of arteries in the circle of Willis. Healthy arterial wall consists of three distinct layers: tunica intima, tunica media and tunica adventitia. Histological aneurysm-related analyses indicate loss of normal layered structure and degradation of extracellular matrix. In IAs, remodelling of medial layer through apoptosis and proliferation of smooth muscle cells (SMCs) has been associated with rupture. These changes cause aneurysm wall to become fragile and lose its elasticity. Rupture occurs when hemodynamic stress exceeds the tensile strength of IA wall. In IAs, changes in hemodynamic forces seem to induce pro-inflammatory signaling and infiltration of leukocytes. In extracranial aneurysms, certain hemodynamic forces have been associated with aneurysm growth. In one embodiment, the vascular inflammation is related to an aneurysm. In one embodiment, the vascular inflammation is intracranial aneurysm. In one embodiment, the vascular inflammation is related to a cerebral aneurysm.

In vascular system, inflammation occurs also in connection with atherosclerosis, strokes, abscesses, infarcts, ischemias and other vascular pathologies such as vasculitides.

Atherosclerosis is a disease in which the lumen of an artery narrows due to the build-up of a lipid-rich plaque in tunica intima. Risk factors of atherosclerosis include abnormal cholesterol levels, high blood pressure, diabetes, smoking, obesity, family history, and an unhealthy diet. The plaque consists of fat, cholesterol, calcium, inflammatory cells and their remnants and other substances found in the blood. Atherosclerosis is associated with inflammatory processes inside the plaque itself and in the endothelial cells of the vessel wall associated with retained low-density lipoprotein (LDL) particles. This retention may be a cause, an effect, or both, of the underlying inflammatory process. In one embodiment, the vascular inflammation is related to atherosclerosis.

A stroke is an acute emergence of neurological symptoms due to cerebrovascular disease, either due to vessel occlusion (ischemic stroke) or vessel rupture (hemorrhagic stroke). The main risk factor for both types of strokes is high blood pressure. Other risk factors include smoking, obesity, high blood cholesterol and diabetes mellitus. In one embodiment, the vascular inflammation is related to a stroke.

Ischemia can be characterized as insufficient supply of oxygen and nutrition to an area of tissue due to a disruption in blood supply. The blood vessel supplying the affected area may be obstructed due to stenosis, thrombosis, embolism or occlusion by other local vascular pathology. In one embodiment, the vascular inflammation is related to ischemia.

Infarction means tissue death due to inadequate blood supply to the affected area. It may be caused by prolonged ischemia. In one embodiment the vascular inflammation is related to infarction.

Vasculitis is inflammation of blood vessels. It causes changes in the blood vessel walls, including thickening, weakening, narrowing or scarring. These changes may restrict blood flow, resulting in organ and tissue damage. There are many types of vasculitides. Vasculitis may be triggered by an infection, such as Herpes simplex virus infection. Vasculitis might affect just one organ, or several organs. The condition can be acute or chronic. In one embodiment, the vascular inflammation is related to vasculitis.

The inflammation in vascular system can be sterile or it can result from infection. In other words, the inflammation in vascular system can be autoimmune or infection driven. An example of inflammation caused by infection is vasculitis caused by herpes simplex virus.

Due to inflammatory changes within the vascular cells, inflammatory factors, which may often be proteins, become expressed or overexpressed in the vascular wall such as the aneurysm wall. These certain proteins can be considered as are biomarkers for inflammation (i.e. inflammatory markers) which are important in the context of the present disclosure. Inflammatory markers presented in the vascular inflammation can be used as anchoring targets or objects for the targeting liposomes via the antibody attached to the targeting liposome, said antibody associated with the inflammatory marker secreted from or presented at the site of the inflammation.

Cyclooxygenase-2 (Cox2) is an enzyme that takes part in synthesis of prostaglandins. It is a well-studied protein and inflammatory marker and target for many pharmaceutical agents such as acetylsalicylic acid (aspirin) and ibuprofen.

The prior research has shown an elevated expression of Cox2 in the IA wall. Upregulation of numerous other proteins is also present in IAs, and thus these other proteins presented in, secreted from or accumulated at the site of IA may be used in a similar manner for anchoring the targeting liposomes to the inflammatory site. In principle, the anchor for the targeting liposomes can be any protein that is abundant in vascular inflammation but scarce in healthy and/or non-inflammatory vasculature. Specifically, the anchor can be any protein that is abundant in aneurysms or other vascular pathologies but scarce in healthy vessels. The inflammatory marker works thus as an anchor for the targeting liposome, through the antibody carried by the targeting liposome, to attach it to the vascular endothelium or other structure or marker in interest.

There are several inflammatory markers related to sterile inflammations as well as inflammations, which result from different infections. In other words, there are several inflammatory markers related to the vascular inflammation which can be either autoimmune or infection driven. An inflammatory marker is an indication and/or a product of a vascular inflammation, which may be imaged with the imaging method according to the present disclosure. In one embodiment, the biological inflammatory marker is an indication and/or a product of vascular inflammation related to an aneurysm or atherosclerosis. In one embodiment, the biological inflammatory marker is an indication and/or a product of vascular inflammation related to a stroke, an abscess, an infarct, an ischemia and/or another vascular pathology such as a vasculitis.

In various embodiments at least one vascular inflammatory marker is secreted from a sterile inflammation.

In various embodiments the inflammation is related to an aneurysm, an atherosclerosis, a stroke, an abscess, an infarct, an ischemia and/or another vascular pathology such as a vasculitis.

In various embodiments the inflammation is resulted from an infection. The infection may be a vasculitis.

The inflammatory markers and other markers related to vascular inflammation The inflammatory markers that are secreted from or accumulated at or presented in vascular inflammation and that can be utilized for anchoring the targeting liposome to the site of the vascular inflammation according to the present disclosure are presented in Tables 1 and 2. The inflammatory marker may also be other than listed in Tables 1 and 2.

In this document by the term inflammatory marker is meant any biological marker which is related to or associated with inflammation. Therefore, in this document the inflammatory marker may be also, among the inflammatory markers, for example an inflammatory mediator, or a marker related to inflammation or another inflammatory factor.

TABLE 1

| | Group (including also the |
|---|---|
| | subgroups, where the |
| | specific marker may be |
| Inflammatory marker | other than mentioned |
| α-Smooth muscle cell actin | Mural cells |
| CD31+ Endothelial cell | Endothelial cells |
| VCAM-1 | Endothelial cells |
| ICAM-1 | Endothelial cells |
| CD34+ Pre-endothelial cell | Neovessels |
| Collagens (I, III, IV, V), fibronectin, laminin | Collagens and their linking |
| | proteins |
| Elastin | Elastin |
| Neutrophil (CD11b, CD16, and CD66b) | Inflammatory cells |
| CD45+ Leukocytes | Inflammatory cells |
| CD163+ Macrophages | Inflammatory cells |
| CD68+ Macrophages | Inflammatory cells |
| CD3+ T Lymphocytes | Inflammatory cells |
| Human leukocyte antigen-DR | Inflammatory cells |
| Tryptase/chymase for mast cells | Inflammatory cells |
| Fibrin | Blood borne/thrombus |
| Plasmin and plasminogen activators | Blood borne/thrombus |
| Glycophorin A for red blood cells | Blood borne/thrombus |
| Serum amyloid A | Blood borne/thrombus |
| CRP | Blood borne/thrombus |
| Apolipoprotein B-100 for VLDL, IDL, | Blood borne/thrombus |
| and LDL | |
| Hydroxynonenal for ox-lipid | Blood borne/thrombus |
| Bacteria | Blood borne/thrombus |
| Monocyte Chemoattractant Protein-1 | Inflammatory mediator |
| Myeloperoxidase | Inflammatory mediator |
| Hemeoxygenase 1 | Inflammatory mediator |
| Prostaglandin E2 Receptor | Inflammatory mediator |
| Cyclo-oxygenase 2 | Inflammatory mediator |
| Complement, C5b9 | Inflammatory mediator |
| Complement C3a, C5a | Inflammatory mediator |
| Interleukins | Inflammatory mediator |
| TNF-a | Inflammatory mediator |
| Matrix metalloproteinase 9 | Degrading enzyme |
| Matrix metalloproteinase 2 | Degrading enzyme |
| Other MMPs | Degrading enzyme |
| Cathepsins (D, G, S, B, K) | Degrading enzyme |
| Neutrophil elastase | Degrading enzyme |
| Adipophilin | Cell signalling protein |
| Growth factor receptors e.g. for VEGF, | Cell signalling protein |
| bFGF, TGF-b, | |

TABLE 2

| | Group (including also |
|---|---|
| | the subgroups, where |
| | the specific marker |
| Inflammatory marker | may be other than |
| IA (or arterial) wall | mentioned in *) |
| α-Smooth muscle cell actin | Smooth muscle cells |
| Myosin heavy chain | Smooth muscle cells |
| Smoothelin | Smooth muscle cells |
| S100A4 | Smooth muscle cells |
| Laminin-1 | Basal lamina |
| Fibronectin | Component of |
| | extracelular matrix |
| Collagens (I, III, IV, V) | Collagens and their |
| | linking proteins |
| CD31+ Endothelial cell | Endothelial cells |
| VCAM-1 | Endothelial cells |
| ICAM-1 | Endothelial cells |
| CD34+ Pre-endothelial cell | Neovessels |
| Connexins (Cx37, Cx40, Cx43) | All cells in the |
| | aneurysm wall |
| Elastin | Elastin |
| Neutrophil (CD11b, CD16, and CD66b) | Inflammatory cells |
| CD45+ Leukocytes | Inflammatory cells |
| CD163+ Macrophages | Inflammatory cells |
| CD68+ Macrophages | Inflammatory cells |
| Other monocyte/macrophage markers (CD4, | Inflammatory cells |
| CD14, CD114, CD11a, CD11b, CD91, CD16) | |
| CD3+ T Lymphocytes | Inflammatory cells |
| Other lymphocyte markers (CD4, CD8, CD19, | Inflammatory cells |
| CD20, CD24, CD25, CD38, CD22) | |
| Natural killer cells (CD16, CD56, | Inflammatory cells |
| CD30, CD38) | |
| Human leukocyte antigen-DR | Inflammatory cells |
| Tryptase/chymase for mast cells | Inflammatory cells |
| Fibrin | Blood borne/thrombus |
| Platelets (CD61) | Blood borne/thrombus |
| Plasmin and plasminogen activators | Blood borne/thrombus |
| Glycophorin A for red blood cells | Blood borne/thrombus |
| Serum amyloid A | Blood borne |
| C-reactive protein | Blood borne |
| Apolipoprotein B-100 for VLDL, IDL, and LDL | Blood borne |
| Apolipoprotein A-1 for HDL | Blood borne |
| Apolipoprotein E | Blood borne |
| ATP-binding cassette transporter | Inflammatory cells |
| Hydroxynonenal for ox-lipid | Blood borne |
| Malondialdehyde for ox-lipid | Blood borne |
| Bacteria including Porfyromonas gingivalis, | Blood borne |
| Fusobacterium nucleatum, Streptococcus | |
| mutans, Agregatibacter actinomycetemcomitans, | |
| Treponema denticola, Prevotella intermedia, | |
| Tannerella forsythia | |
| Lipopolysaccharides | Blood borne |
| Monocyte Chemoattractant Protein-1 | Inflammatory mediator |
| Myeloperoxidase | Inflammatory mediator |
| Hemeoxygenase 1 | Inflammatory mediator |
| Prostaglandin E2 Receptor | Inflammatory mediator |
| Cyclo-oxygenase 2 | Inflammatory mediator |
| Complement, C5b9 | Inflammatory mediator |
| Complement C3a, C5a | Inflammatory mediator |
| Interleukins (IL2, IL6) | Inflammatory mediator |
| Other interleukins (IL1-36) | Inflammatory mediator |
| TNF-a | Inflammatory mediator |
| Matrix metalloproteinase 9 | Degrading enzyme |
| Matrix metalloproteinase 2 | Degrading enzyme |
| Other MMPs (1-28) | Degrading enzyme |
| Cathepsins (D, G, S, B, K) | Degrading enzyme |
| Neutrophil elastase | Degrading enzyme |
| Adipophilin | Cell signaling protein |
| Growth factor receptors e.g. for VEGF, bFGF, | Cell signaling protein |
| TGF-b, | |

In one embodiment, the inflammatory marker is a cytokine, such as tumor necrosis factor alpha (TNF-α), tumor necrosis factor beta (TNIβ-), interferon gamma (INF-γ), interleukin IL-1α, interleukin IL-1β or interleukin IL-18.

In one embodiment, the inflammatory marker is a chemokine, such as a monocyte-chemoattractant protein-1 (MCP-1).

In one embodiment, the inflammatory marker is transcription Factor Nuclear Factor-kappa B (NFκB), the vascular cell adhesion molecule-1 (VCAM-1), anaphylatoxin C3a, anaphylatoxin C5a.

In one embodiment, the inflammatory marker or a marker related to the pathology in interest is a receptor expressed by a cell in the aneurysm wall or in the other vascular pathology in interest like CD163.

In one embodiment, the inflammatory marker is cyclooxygenase-2 (Cox2).

Generally, an antibody is an immunoglobulin molecule, or domain of said molecule which comprises an antigen binding site that forms noncovalent bonds with antigen (such as inflammatory marker). The amount of interaction affects the affinity of certain antibodies to certain antigens. Due to their capability to bind with variable affinity to epitope regions of the specific antigens, antibodies have numerous scientific, diagnostic and therapeutic applications. There are a number of different antibodies commercially available.

In one embodiment, the vascular inflammation is present in an aneurysm, and wherein the antibody is selected from a list of antibodies against vascular inflammatory markers presented in Table 1.

In one embodiment, the antibody is selected from the list of antibodies against vascular inflammatory markers presented in Table 1.

In one embodiment the targeting liposome for use in the diagnosis or imaging of aneurysm comprises antibodies against at least one vascular inflammatory marker associated with aneurysm and a label and/or a contrast agent. In an embodiment the at least one vascular inflammatory marker comprises α-Smooth muscle cell actin. In another embodiment the at least one vascular inflammatory marker is selected from the group consisting of CD31+ Endothelial cell, Cyclo-oxygenase 2, α-Smooth muscle cell actin and CD45+ leukocytes. Still in another embodiment the at least one vascular inflammatory marker is selected from the group consisting of a-smooth muscle cell actin, myosin heavy chain, smoothelin, S100A4, laminin-1, fibronectin, collagens (I, III, IV, V), CD31+ endothelial cell, vascular cell adhesion molecule 1, intercellular adhesion molecule 1, CD34+ pre-endothelial cell, connexins (Cx37, Cx40, Cx43), elastin, neutrophil (CD11b, CD16, and CD66b), CD45+ leukocytes, CD163+ macrophages, CD68+ macrophages, monocyte/macrophage markers (CD4, CD14, CD114, CD11a, CD11b, CD91, CD16), CD3+ T-lymphocytes, lymphocyte markers (CD4, CD8, CD19, CD20, CD24, CD25, CD38, CD22), natural killer cells (CD16, CD56, CD30, CD38), human leukocyte antigen-DR, tryptase/chymase+ mast cells, fibrin, platelets (CD61), plasmin and plasminogen activators, glycophorin A+ red blood cells, serum amyloid A, C-reactive protein, apolipoprotein B-100+ very low density lipoprotein, intermediate density lipoprotein, and low density lipoprotein, apolipoprotein A-1+ high density lipoprotein, apolipoprotein E, ATP-binding cassette transporter, hydroxynonenal+ oxidized lipid, malondialdehyde+ oxidized-lipid, bacteria or bacterial fragments of Porfyromonas gingivalis, Fusobacterium nucleatum, Streptococcus mutans, Agregatibacter actinomycetemcomitans, Treponema denticola, Prevotella intermedia, and/or Tannerella forsythia, lipopolysaccharides, monocyte chemoattractant protein-1, myeloperoxidase, hemeoxygenase 1, prostaglandin E2 receptor, cyclo-oxygenase 2, complement proteins (C5b9, C3a, C5a), interleukins (IL2, IL6, IL1-36), tumor necrosis factor alpha, matrix metalloproteinase 9, matrix metalloproteinase 2, matrix metalloproteinases (1-28), cathepsins (D, G, S, B, K), neutrophil elastase, adipophilin, growth factors (vascular endothelial growth factor, basic fibroblast growth factor, transforming growth factor beta) and their receptors.

In one embodiment the targeting liposome for use in the diagnosis or imaging of aneurysm comprises a label and/or contrast agent comprising a moiety encapsulated (including being held in the membrane of the liposome) into the targeting liposome, which moiety is selected from a magnetic moiety, a radioactive moiety, a radionuclide moiety, a luminescent moiety and a fluorescent moiety.

In one embodiment of a targeting method for anchoring the targeting liposome, the liposome carries antibodies for a vascular inflammatory marker associated with aneurysm and a label and/or a contrast agent to an intended tissue area secreting at least one vascular inflammatory marker associated with aneurysm, the method comprises administering the liposome to a subject. In an embodiment the at least one vascular inflammatory marker comprises α-Smooth muscle cell actin. In another embodiment the at least one vascular inflammatory marker is selected from the group consisting of CD31+ Endothelial cell, Cyclo-oxygenase 2, α-Smooth muscle cell actin and CD45+ leukocytes. Still in another embodiment the at least one vascular inflammatory marker is selected from the group consisting of α-smooth muscle cell actin, myosin heavy chain, smoothelin, S100A4, laminin-1, fibronectin, collagens (I, Ill, IV, V), CD31+ endothelial cell, vascular cell adhesion molecule 1, intercellular adhesion molecule 1, CD34+ re-endothelial cell, connexins (Cx37, Cx40, Cx43), elastin, neutrophil (CD11b, CD16, and CD66b), CD45+leukocytes, CD163+ macrophages, CD68+ macrophages, monocyte/macrophage markers (CD4, CD14, CD114, CD11a, CD11b, CD91, CD16), CD3+ T-lymphocytes, lymphocyte markers (CD4, CD8, CD19, CD20, CD24, CD25, CD38, CD22), natural killer cells (CD16, CD56, CD30, CD38), human leukocyte antigen-DR, tryptase/chymase+ mast cells, fibrin, platelets (CD61), plasmin and plasminogen activators, glycophorin A+ red blood cells, serum amyloid A, C-reactive protein, apolipoprotein B-100+ very low density lipoprotein, intermediate density lipoprotein, and low density lipoprotein, apolipoprotein A-1+ high density lipoprotein, apolipoprotein E, ATP-binding cassette transporter, hydroxynonenal+oxidized lipid, malondialdehyde+ oxidized-lipid, bacteria or bacterial fragments of Porfyromonas gingivalis, Fusobacterium nucleatum, Streptococcus mutans, Agregatibacter actinomycetemcomitans, Treponema denticola, Prevotella intermedia, and/or Tannerella forsythia, lipopolysaccharides, monocyte chemoattractant protein-1, myeloperoxidase, hemeoxygenase 1, prostaglandin E2 receptor, cyclo-oxygenase 2, complement proteins (C5b9, C3a, C5a), interleukins (IL2, IL6, IL1-36), tumor necrosis factor alpha, matrix metalloproteinase 9, matrix metalloproteinase 2, matrix metalloproteinases (1-28), cathepsins (D, G, S, B, K), neutrophil elastase, adipophilin, growth factors (vascular endothelial growth factor, basic fibroblast growth factor, transforming growth factor beta) and their receptors.

In this document, the term “imaging” or “clinical imaging” refers to the use of any imaging method to visualize a structure, e.g., a blood vessel, a capillary, blood pool, inflammation, or plaque, either in vivo or ex vivo by measuring the differences in absorption of energy transmitted by or absorbed by the tissue. Imaging method includes magnetic resonance imaging (MRI), magnetic resonance angiography (MRA), x-ray imaging, computed tomography (CT), computed tomography angiography (CTA), positron emission tomography (PET), single-photon emission computed tomography (SPECT), Digital subtraction angiography (DSA), and the like.

Magnetic resonance angiography (MRA) and computed tomography angiography (CTA) are examples of non-invasive imaging methods used in diagnosis of blood vessel diseases or related conditions, such as aneurysms or occlusions or other pathologies. Generally, CTA uses an injection of contrast material into blood vessels of a subject and CT scanning to help diagnose and evaluate blood vessel disease or related conditions, such as aneurysms or blockages. MRA has benefits over CT angiography as it doesn't produce ionizing radiation. Generally, contrast enhanced MRA uses gadolinium-based agents or the like to increase T1 signal in images and produce more accurate information about the vasculature of a subject.

Positron emission tomography (PET) is an imaging method that uses radioactive contrast material to visualize and measure different processes in a subject. Different contrast agents, tracers and/or labels are used for various imaging purposes, depending on the target process within the body of the subject. Fluorodeoxyglucose (FDG) conjugated with fluorine-18 (¹⁸F) is the most commonly used contrast material for PET imaging. The concentrations of imaged FDG or other contrast material indicate tissue metabolic activity as it corresponds to the regional uptake of the contrast material.

Single-photon emission computed tomography (SPECT) is an imaging method utilizing gamma rays for providing 3D information of a subject. The method needs delivery of a gamma-emitting radioisotope into a subject, normally through injection into the bloodstream. The radioisotopes typically used in SPECT as contrast agents, labels and/or tracers are iodine-123, technetium-99m, xenon-133, thallium-201, fluorine-1 and a gallium(III) isotope. Generally, the marker radioisotope is attached to a specific ligand to create a radioligand, whose properties bind it to certain types of tissues.

Digital subtraction angiography (DSA) is an invasive fluoroscopic imaging method used for visualizing blood vessels and in diagnosis of vascular diseases. Radiopaque structures such as bones are subtracted digitally from the image, thus allowing accurate depiction of the lumen of the blood vessels.

In the present disclosure, the imaging method as well as the label and/or contrast agent are selected from the methods known in the art to be capable for vascular imaging.

Aneurysms including intracranial aneurysms can be treated by open microsurgery (clipping or revascularization) or endovascular (coiling or stenting) methods. Depending on biology of the aneurysm wall and the character of the inflammation the chosen treatment option might have severe effects. These effects can be, for example, a residual aneurysm formed after open microsurgery or a recanalization or residual aneurysm caused by an endovascular treatment.

For detecting what kind of inflammatory processes are ongoing in the specific aneurysm, including what kind of vascular inflammatory markers secreted from the aneurysm, and further for selecting the best treatment option based on said detecting of inflammatory processes for aneurysms are lacking in the art.

In one embodiment the imaging method further comprises selecting a treatment option, which treatment option can be selected from the list consisting of clipping, coiling, stenting, and revascularization, for the detected aneurysm is based on which antibody carried in the targeting liposome is bound to the at least one vascular inflammatory marker secreted from the detected tissue area. This provides an opportunity to detect more precise form of the inflammation or pathways of the inflammation associated with a specific aneurysm, and hence, it gives an opportunity to select a correct treatment option for the aneurysm in question to avoid a residual or recanalization of the treated intracranial aneurysm. This method can further comprise a plurality of imaging phases making possible to improving of specifying different inflammatory markers associated with the aneurysm in question, where in the method:

a. in the first imaging phase the intended tissue secreting at least one vascular inflammatory marker associated with aneurysm is detected by using a targeting liposome comprising at least two, preferably at least four, more preferably at least six, different antibodies against some vascular inflammatory markers secreted from the detected aneurysm, which markers are disclosed in this document, and

b. in at least one subsequent imaging phase the intended tissue secreting at least one vascular inflammatory marker associated with aneurysm is detected by using another targeting liposome that is devoid of at least one antibody compared to the targeting liposome used in the preceding imaging phase.

In various embodiments the targeting liposome comprises at least one type of lipid and at least one type of antibody against at least one inflammatory marker, and at least one label and/or a contrast agent capable of enhancing imaging contrast in the imaging method known in the art.

In various embodiments, at least one type of lipids of the targeting liposome according to the present disclosure, is selected from the group of phosphatidylcholides, phosphatidylethanolamines, phosphatidylserines, phosphatidylglycerols, lipids comprising polyethylene glycol (i.e., pegylated lipids), pegylated phospholipids, ceramides, sphingolipids, fatty acids and cholesterol.

At least some of the lipids of the targeting liposome are capable to create a spherical vesicle having at least one lipid bilayer. Said liposome bilayer is formed into an isolated environment where molecules, e.g., label and/or contrast agents, can be encapsulated inside.

The targeting liposome may comprise at least one lipid selected from the group comprising 1,2-dipalmitoyl-sn-glycero-3-phosphocholine (DPPC), 1,2-distearoyl-sn-glycero-3-phosphoethanolamine-N-[amino(polyethylene glycol)-2000] (DSPE-PEG2000), maleimide derivatized DSPE-PEG2000 (DSPE-PEG2000-Mal), 1,2-dipalmitoyl-sn-glycero-3-phosphoethanolamine (DPPE), rhodamine-labeled phosphatidyl ethanolamine (Rh-DPPE), and cholesterol (CHOL).

In various embodiments, the at least one type of lipid is phospholipid derivative.

In one embodiment, the phospholipid derivative is dipalmitoyl-phosphotidyl-choline (DPPC). In one embodiment, the phospholipid derivative is distearoyl-phosphoethanolamine [methoxy poly(ethylene glycol)-2000] (mPEG2000-DSPE).

In one embodiment, said at least one lipid bilayer of the targeting liposome comprises at least one first lipid or phospholipid, at least one second lipid or phospholipid, and at least one third lipid or phospholipid derivative.

In one embodiment, the targeting liposome comprises at least one first lipid or phospholipid, at least one second lipid or phospholipid, and at least one third lipid or phospholipid derivative.

The targeting liposome may comprise a linker lipid or phospholipid that is attached to the outer surface of the targeting liposome. Furthermore, the antibody may be attached to the targeting liposome via the linker lipid or phospholipid.

In one embodiment, the targeting liposome comprises at least one first lipid or phospholipid, at least one second lipid or phospholipid, at least one third lipid or phospholipid derivative, and a linker lipid or phospholipid. The linker lipid or phospholipid is attached to the outer surface of the liposome of the targeting liposome.

The at least one first lipid or phospholipid may be present from the total lipid amount of the targeting liposome in the amount of about 55 to 92.5 mol %, more preferably in the amount of about 65 to 85 mol %, even more preferably in the amount of about 70 to 85 mol %, and the most preferably in the amount of about 75 to 85 mol %. Furthermore, the at least one second lipid or phospholipid is present from the total lipid amount of the targeting liposome in the amount of about 4 to 25 mol %, more preferably in the amount of about 7.5 to 22.5 mol %, even more preferably in the amount of about 8.5 to 20 mol %, and the most preferably in the amount of about 15 to 20 mol %. Furthermore, the at least one third lipid or phospholipid is present from the total lipid amount of the targeting liposome in the amount of about 2.5 to 15 mol %, more preferably in the amount of about 3.5 to 12.5 mol %, even more preferably in the amount of about 4.0 to 10 mol %, and the most preferably in the amount of about 4 to 6 mol %. Furthermore, the linker lipid or phospholipid is present from the total lipid amount of the targeting liposome in the amount of about 0.5 to 12.5 mol %, more preferably in the amount of about 1 to 10 mol %, even more preferably in the amount of about 0.5 to 7.5 mol %, and the most preferably in the amount of about 0.5 to 2 mol %.

In one embodiment, the at least one first lipid or phospholipid is DPPC, the at least one second lipid or phospholipid is cholesterol, the at least one third lipid or phospholipid derivative is mPEG2000-DSPE, and the linker lipid or phospholipid is Mal-PEG2000-DSPE.

In one embodiment, the at least one first lipid or phospholipid is DPPC and is present from the total lipid amount of the targeting liposome in the amount of about 80 mol %, the at least one second lipid or phospholipid is cholesterol and is present from the total lipid amount of the targeting liposome in the amount of about 10 mol %, the at least one third lipid or phospholipid derivative is mPEG2000-DSPE and is present from the total lipid amount of the targeting liposome in the amount of about 5 mol %, and the linker lipid or phospholipid is Mal-PEG2000-DSPE and is present from the total lipid amount of the targeting liposome in the amount of about 5 mol 15%.

In one embodiment, the at least one first lipid or phospholipid is DPPC, the at least one second lipid or phospholipid is DPPE, the at least one third lipid or phospholipid derivative is mPEG2000-DSPE, and the linker lipid or phospholipid is Mal-PEG2000-DSPE.

In one embodiment, the at least one first lipid or phospholipid is DPPC and is present from the total lipid amount of the targeting liposome in the amount of about 90 mol %, the at least one second lipid or phospholipid is DPPE and is present from the total lipid amount of the targeting liposome in the amount of about 5 mol %, the at least one third lipid or phospholipid derivative is mPEG2000-DSPE and is present from the total lipid amount of the targeting liposome in the amount of about 4 mol %, and the linker lipid or phospholipid is Mal-PEG2000-DSPE and is present from the total lipid amount of the targeting liposome in the amount of about 1 mol %.

In various embodiments the linker lipid is phospholipid derivative.

In one embodiment, the linker lipid is Mal-PEG2000-DSPE.

In one embodiment, the targeting liposome comprises the following phospholipids: Dipalmitoyl-phosphotidyl-choline (DPPC), phosphatidylcholine from egg, chicken (eggPC), dipalmitoyl-sn-glycero-3-phosphoethanolamine (DPPE) distearoyl-phosphoethanolamine [methoxy(poly-ethylene glycol)-2000] (mPEG2000-DSPE), maleimide derivatized PEG2000-DSPE (mal-PEG2000-DSPE), distearoyl-phosphoethanolamine [carboxy(polyethylene glycol)-2000] (carboxy PEG2000-DSPE), rhodamine-labeled phosphatidyl dipalmitoyl ethanolamine (Rh-PE) and cholesterol (CHOL). The range of molar ratio of lipids can be as follows: DPPC or eggPC—70-90%; CHOL—10-40%; DPPE—10-20%; mPEG2000-DSPE — 4-10%; mal-PEG2000-DSPE — 1-5%; carboxy-PEG2000-DSPE—1-5%; Rh-PE—0.5-1%. This content of lipids and their molar ratios ensure a stabile lipid structure for the targeting liposome suitable to be used in vivo imaging.

In one embodiment, the lipid content of the targeting liposome consists of the following phospholipids: Dipalmitoyl-phosphotidyl-choline (DPPC), phosphatidylcholine from egg, chicken (eggPC), dipalmitoyl-sn-glycero-3-phosphoethanolamine (DPPE) distearoyl-phosphoethanolamine [methoxy(poly-ethylene glycol)-2000] (mPEG2000-DSPE), maleimide derivatized PEG2000-DSPE (mal-PEG2000-DSPE), distearoyl-phosphoethanolamine [carboxy(polyethylene glycol)-2000] (carboxy PEG2000-DSPE), rhodamine-labeled phosphatidyl dipalmitoyl ethanolamine (Rh-PE) and cholesterol (CHOL).

In some embodiments, the targeting liposome has an average diameter between about 70 and 250 nm.

In some embodiments, the targeting liposome has an average diameter of less than about 150 nm.

In some embodiments, the targeting liposome has an average diameter of more than about 80 nm.

In some embodiments, the targeting liposome has an average diameter between about 80 and 150 nm.

In some embodiments, the targeting liposome has an average diameter between about 30 and 90 nm.

In some embodiments, the targeting liposome has an average diameter between about 90 and 120 nm.

In some embodiments, the targeting liposome has an average diameter is about 100 nm.

In some embodiments, the targeting liposome has an average diameter is about 90 nm.

In some embodiments, the targeting liposome has an average diameter is about 80 nm.

In some embodiments, the at least one type of lipid is capable of self-assembling into an amphiphilic colloidal form of the liposome.

In some embodiments, the liposome comprises at least one polymeric excipient capable of stabilizing the amphiphilic colloidal form of the targeting liposome.

In some embodiments, the at least one polymeric excipient is a derivative of polyethylene glycol (PEG).

In one embodiment, the targeting liposome comprises antibodies against at least one inflammatory marker.

In one embodiment, the targeting liposome comprises antibodies against one inflammatory marker.

In one embodiment, the targeting liposome comprises antibodies against two separate inflammatory markers.

In one embodiment, the targeting liposome comprises antibodies against three or more different inflammatory markers.

In some embodiments, the at least one antibody is against Anti-cyclooxygenase-2 (Anti-Cox2).

In some embodiments, the at least one antibody marker is against Immunoglobulin G (igG).

In some embodiments, the at least one antibody is against hen lysozyme.

In some embodiments, the at least one antibody is against alpha-smooth muscle cell actin (aSMA).

The targeting liposome may typically encapsulate or associate a contrast agent and/or label. It should be noted that for purposes of the present disclosure, the identity of the label or contrast agent is not of substantial importance. In other words, for purposes of the present disclosure, the targeting liposome will be utilized similar manner regardless of the label and/or contrast agent used. However, suitable contrast agents and labels may include, for example, fluorescent dyes, such as, for example, fluorescein iso-thiocynate (FITC) and rhodamine; CT contrast agents including iodinated compounds such asiohexol, iodixanol, and iotrolan; and MRI contrast agents including lanthanide aminocarboxylate complexes such as Gadolinium (III) DTPA, Gd-DOTA, Gd-DOTAP, and Gd-DOTMA.

In various embodiments, the label and/or contrast agent comprise a moiety encapsulated into the liposome, which moiety is selected from a magnetic moiety, a radioactive moiety, a radionuclide moiety, a luminescent moiety and a fluorescent moiety.

The targeting liposome may comprise an active pharmaceutical ingredient (API). In some embodiments, the API is capable of producing an intended effect on an inflammation of the subject, where said inflammation is associated with the at least one vascular inflammatory marker secreted from said inflammation.

In one embodiment, the API prevents and/or inhibits blood clotting and/or immunological reactions.

In some embodiments, the targeting liposome comprises a conjugate having the connection between the antibody and API via the linker molecule.

In some embodiments, the API is co-encapsulated with the label and/or contrast agent into the targeting liposome.

The targeting liposome or the composition comprising a targeting liposome according to the present disclosure may be delivered to a subject utilizing any applicable administration method and/or device known in the art, as by injection, for example. One preferred method of administration is injection. One preferred method of administration is intravenous.

Regardless of the administration method or route used the liposome or the composition comprising the targeting liposomes, which may be used in a suitable hydrated form, are formulated into pharmaceutically acceptable dosage forms by conventional methods known in the art.

An effective amount of the liposome according to the present disclosure is generally an amount such that when administered in a physiologically tolerable composition is sufficient to capable enhanced or improved detection or imaging of vascular sites, e.g., site of inflamed blood vessel, atherosclerotic plaque, aneurysm or a lesion the like or other vascular pathology, within the subject.

The present disclosure also relates to a method of imaging a vascular inflammation in a subject, who has been injected with a composition comprising targeting liposomes according to the present disclosure, which targeting liposome carries antibodies against at least one inflammatory marker and at least one label and/or contrast agent. The method comprises scanning the biological activity of the vascular inflammation using an imaging method that detects the label and/or contrast agent carried by the targeting liposome.

The subject may be a human or an animal.

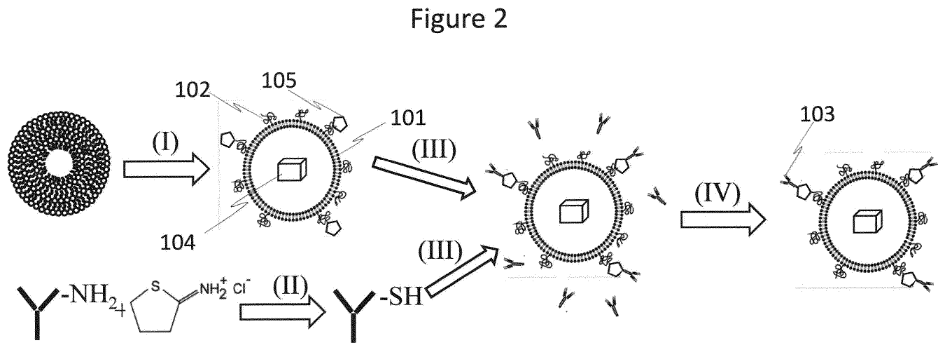

FIG. 2 shows a scheme of the main steps of synthesis of the targeting liposome according to an embodiment, wherein the synthesis comprises the steps of:

I. conversion of multilamellar liposomes to unilamellar liposomes;

II. modification of primary amines of the antibody to be attached to the maleimide group of the linker lipid or linker phospholipid locating at the outer surface of the unilamellar liposome, i.e., targeting liposome, by 2′-iminothilane or Traut's reagent;

III. conjugation of modified proteins of the antibody to the targeting liposome via the maleimide group of the linker lipid or phospholipid; and

IV. separation of unconjugated antibodies from the targeting liposomes comprising conjugated antibodies by ultracentrifuge.

### EXAMPLES

The following examples are given to further illustrate the invention without, however, restricting the invention thereto.

### Example 1—Creating of Targeting Liposomes

A lipid film containing 90% DPPC, 5% DPPE and 5% DSPE-PEG2000 was hydrated in PBS buffer solution containing 10 mM concentration of the dye, 5(6) carboxyfluorescein. Chelating agent EDTA was also added to buffer solution to get rid of calcium ions. PEG-lipids were used to prepare the formed large unilamellar vesicles (LUVs) to be suitable for an immune system. LUV formation was confirmed with dynamic light scattering. Non-encapsulated carboxyfluorescein was removed by passing the sample through 3 Sephadex G50 filters. Encapsulation was confirmed by measuring 40% increase in fluorescence after addition of Triton-X detergent. This experiment was made several times with different LUV concentrations ranging from 1 mM to 10 mM. LUVs containing dye were then concentrated in a centrifuge by using Vivaspin filters. Lipid concentration was measured with the Bartlett assay by quantifying amount of inorganic phosphate in sample.

After encapsulation was proven successful, proteins were added on the surface of liposomes. Hen lysozyme was used as a model protein to establish the protocol and to characterize conjugation of the protein to the produced targeting liposomes. 1% of DSPE-PEG2000 maleimide (DSPE-PEG2000-Mal) was added to the composition of formed liposomes to attach antibodies to the formed liposomes via the PEG-polymers that contain a maleimide.

Lysozyme was then incubated with Traut's reagent to open disulfide bonds and coupled with maleimide containing LUVs. Sample was incubated in room temperature for 3 hours allowing bonds to form between maleimide and thiol groups of the protein. Excess protein was separated from the sample by using sephadex G50 gel filtration. Liposome integrity was confirmed with dynamic light scattering and fluorescence spectroscopy measurements of encapsulated carboxyfluorescein. SDS-PAGE electrophoresis was used to confirm the attachment of lysozyme. Biorad mini-protean stain free gel was used as it contains trihalo compounds that enhance tryptophan fluorescence visualizing the proteins after UV-light incubation.

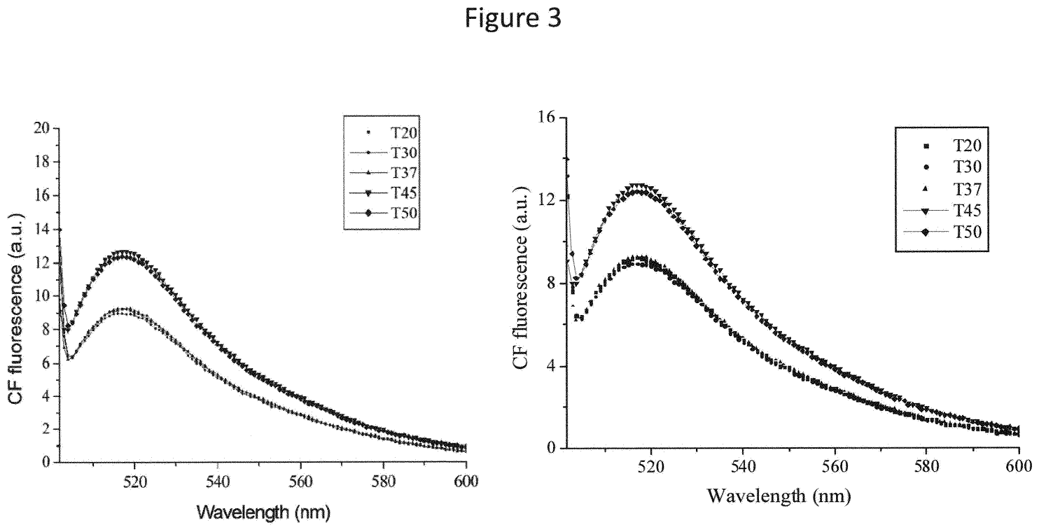

FIG. 3 shows measured fluorescence spectra of the targeting liposomes with encapsulated carboxyfluorescein (CF), indicating that integrity of the targeting liposomes is retained during the experiments.

### Example 2—The Principles of Selection and Engineering of Targeting Liposomes

### Selection Criteria

Anti-COX-2 antibody has been selected as COX-2 is one of the inflammatory markers in labile aneurysm and its presence is significantly increased in aneurysms before their rupture.

Anti-aSMA antibody for smooth muscle cells has been selected as lack of smooth muscle cells related to inflammatory milieu is seen in ruptured and rupture prone aneurysms.

Liposomes consist of the following phospholipids: 1,2-dipalmitoyl-sn-glycero-3-phosphocholine (DPPC), 1,2-distearoyl-sn-glycero-3-phosphoethanolamine-N-[amino(polyethylene glycol)-2000] (DSPE-PEG2000), maleimide derivatized DSPE-PEG2000 (DSPE-PEG2000-Mal), 1,2-dipalmitoyl-sn-glycero-3-phosphoethanolamine (DPPE), rhodamine-labeled phosphatidyl ethanolamine (Rh-DPPE), and cholesterol (CHOL). All of them are commercially available.

Phase transition of DPPC is 41° C., so using DPPC as the core lipid provides thermal stability of liposomes at physiological temperatures. Cholesterol is known as a stiffness regulator and stabilizer of lipid bilayer. Therefore, the use of cholesterol gives additional stability to liposomes. Pegylation of phospholipids creates a protective layer making liposomes less visible for reticular endothelial system, thus prolonging the circulation of targeting liposomes when injected into a subject.

### Preparation of Liposomes

PEGylated liposome containing DPPC, CHOL, and DSPE-PEG2000 in a molar ratio of 80:10:10 was prepared using the thin film hydration method. Phospholipids (DPPC and DSPE-PEG2000), CHOL, and carboxyfluorescein (as a fluorescence label) were solubilized in chloroform in a round bottomed flask and dried to form a thin lipid film, first, under nitrogen stream and, second, under vacuum to eliminate traces of chloroform. As a linker lipid or phospolipid, DSPE-PEG2000-Mal was added into the liposome suspension with the molar ratio of DPPC: CHOL: DSPE-PEG2000: DSPE-PEG2000-Mal=80:10:5:5. Then, the lipid film was hydrated with phosphate buffered saline (PBS, pH 7.4) containing 0.1 m EDTA for 1-2 h. Afterwards, the resultant multilamellar dispersions were extruded by using polycarbonate membrane filter with pore size 0.1 mm to produce so called large unilamellar vesicles (LUVs)

For confocal microscopy, 0.5 mol % of Rh-DPPE relative to total lipids (i.e., DPPC and DSPE-PEG2000) was added to the liposome formulation.

Unentrapped carboxyfluorescein was removed through by Sephadex G25 column with PBS and 0.1 M EDTA applying gravity protocol.

The final PEGylated liposome particles were stored in dark containers at 4-8° C.

### Antibody Conjugation to Liposomes

Conjugation of antiCOX2 to the prepared PEGylated liposome was based on disulfide modification by Traut's reagent and formation —SH groups on the surface of antibody molecule and subsequent linkage to maleimide moiety at PEG2000-DSPE in the liposome. Antibody was thiolated with 2-iminothiolane in PBS and 0.1M EDTA at a molar ratio of 2-iminothiolane: antibody of 50:1 for 2 h at room temperature (RT). Unreacted 2-iminothiolane was removed by a Sephadex G-25 gel column with PBS and 0.1M EDTA. Then, the thiolated antiCOX2 was immediately incubated with the PEGylated liposome containing maleimide at 4-8° C. during 12 h or overnight for preparation of Ab-LUVs.

To remove non-linked antibodies the solution was then subjected to an ultracentrifuge and spinned at 100,000 g for 2 hours. Supernatants from the centrifuged solution were carefully removed and the separated pellets were rehydrated in PBS and EDTA buffer during 4 hours at 4-6° C. with gentle stirring. Re-dissolved pellet contained conjugated targeting liposomes.

The corresponding actions as described above to conjugate antiCOX2 were employed for aSMA conjugation to the produced targeting liposomes.

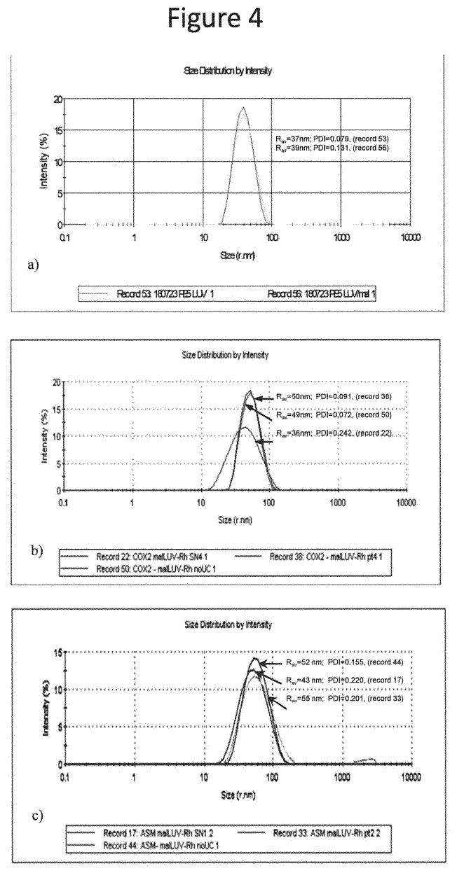

FIG. 4a shows dynamic light scattering (DLS) spectra depicting the particle size distributions of the formed targeting liposomes before and after the addition of the linker lipid or phospholipid of Mal-PEG2000-DSPE, indicating clearly that the addition does not affect size and polydispersity of the liposomes.

FIG. 4b shows DLS for the formed targeting liposomes with Anti-COX2 and ASMA attached before ultracentrifugation for pellets and supernatants.

FIG. 4c shows DLS for the formed targeting liposomes with Anti-COX2 and ASMA attached after ultracentrifugation for pellets and supernatants.

### Example 3

### Preparation of Liposomes

Concentrated lipid solutions were prepared by dissolving lipid powder in chloroform and stored in the freezer (−20° C.). Liposomes consist of the following phospholipids:

Dipalmitoyl-phosphotidyl-choline (DPPC), phosphatidylcholine from egg, chicken (eggPC), dipalmitoyl-sn-glycero-3-phosphoethanolamine (DPPE) distearoyl-phosphoethanolamine [methoxy(poly-ethylene glycol)-2000] (mPEG2000-DSPE), maleimide derivatized PEG2000-DSPE (mal-PEG2000-DSPE), distearoyl-phosphoethanolamine [carboxy(polyethylene glycol)-2000] (carboxy PEG2000-DSPE), rhodamine-labeled phosphatidyl dipalmitoyl ethanolamine (Rh-PE) and cholesterol (CHOL). DPPC, eggPC, DPPE, CHOL and Rh-PE were purchased from Avanti Polar Lipids. mPEG2000-DSPE and mal-PEG2000-DSPE were purchased from Quanta BioDesign. All lipids were of high, more than 96% analytical grade.

Liposomes were prepared using a Hamilton glass syringe to aliquot the corresponding amounts of lipids in a round bottomed flask. The range of molar ratio of lipids were used as follows: DPPC or eggPC—70-90%; CHOL—10-40%; DPPE—10-20%; mPEG2000-DSPE—4-10%; mal-PEG2000-DSPE—1-5%; carboxy-PEG2000-DSPE—1-5%; Rh-PE—0.5-1%. After mixing desired amounts of lipids, they were dried under nitrogen gas stream at room temperature in the dark until chloroform was evaporated and lipid film was formed. To completely remove traces of chloroform, the lipid films were placed under vacuum for at least 4 hours or overnight. Then, to make multilamellar dispersions, the lipid film was hydrated with 10 mM phosphate buffered saline (PBS, pH 7.4) containing 1-5mM EDTA for 1-2 h. The hydration step was performed at the temperature above phase transition of lipids in liposomes determined by presence of DPPC or eggPC and set in the range 41-50° C.

To produce large unilamellar vesicles (LUVs), the resultant multilamellar solutions were passed 20-25 times through polycarbonate membrane filter with pore sizes of 100 nm in the extruder LiposoFast from Avestin under pressure 30-45PSI with an optimal value at 40 PSI. Extrusion was performed in water bath at temperature set above phase transition, in the range of 41-50° C.

The size and polydispersity of liposomes were assessed by utilizing a Malvern Dynamic Light Scattering Instrument. The instrument was checked with polystyrene beads of 100 nm. The diameter obtained is in the range 70-150 nm with polydispersity index varied from 0.02 to 0.3 with optimal values being 100 nm and 0.05 respectively. The optimal size of liposome is around 100 nm. Particles with smaller size are less efficiently opsonized by cells, while liposomes of bigger size are more rapidly removed from the body by the reticuloendothelial system. Polydispersity index is needed to ensure a specific payload of label or contrast agent is present in order to determine the amount of inflammatory biomarker.

Obtained liposomes were kept in dark glass vials and stored at 4° C.

### Antibody Conjugation to Liposomes

Anti-COX-2 antibody has been selected as COX-2 is one of the inflammatory markers in labile aneurysm and its presence is significantly increased in aneurysms before their rupture.

Anti-aSMA antibody for smooth muscle cells has been selected as lack of smooth muscle cells related to inflammatory milieu is seen in ruptured and rupture prone aneurysms.

Anti-CD31+ endothelial and anti-CD45+ leukocytes have been chosen to demonstrate that methods used for targeting are applied on other antibodies.

### Antibody Conjugation to Liposomes

### Method 1.

Conjugations of anti-ASMA (alpha smooth muscle actin antibody (anti-αSMA-Ab)), anti-CD31+ endothelial cell, and anti-CD45+ leukocytes antibodies to the prepared maleimide containing liposome were based on disulfide modification of protein by Traut's reagent and formation —SH groups on the surface of antibody molecule. Sulfhydryl group, also called “thiol group”, specifically reacts with maleimide group and thus provides the linkage to maleimide moiety at PEG2000-DSPE in the liposome. The antibody is first thiolated with 2-iminothiolane at a molar ratio of 2-iminothiolane to antibody in the range of 10-50:1 for 1-3 h at room temperature (RT). Thiolation reaction is efficient in the pH range 7.0-8.0. So, 10-20 mM Phosphate buffer saline and 10-20 mM Hepes buffer saline were used in the thiolation procedure. Additionally, 1-5 mM EDTA was included to reaction buffer to chelate divalent ions and therefore preserve sulfhydrils from oxidation.

Secondly, the unreacted 2-iminothiolane was removed using a desalting gel column Sephadex G-25 equilibrated by PBS and 1-5 mM EDTA buffer. Lastly, to prepare antibody-linked liposomes, the thiolated antibodies were immediately mixed with the PEGylated liposome containing maleimide and incubated 12-18 hours at 4-8° C. To remove non-linked antibodies the solution was then subjected to an ultracentrifuge and spinned at 100,000 g for 1-3 hours. The supernatant was carefully removed. Desired volumes of PBS were added to pellets and they were rehydrated during at least 3 hours or overnight at 4-6° C. with gentle stirring. Re-dissolved pellets contain antibody conjugated LUVs and can be used in further tests.

### Method 2.

Conjugation of anti-ASMA antibodies to the prepared PEGylated liposome containing maleimide was based on disulfide modification of protein via reaction with first 3-(2-pyridyl dithio) propionic acid N-hydroxysuccinimide ester (SPDP) followed by reaction with dithiothreitol (DTT).

Antibody was reacted with SPDP in a 5-10:1 SPDP:protein molar ratio for 30 minutes. Non-reacted SPDP was separated from pyridyldithiol-modified protein by using a Sephadex G-25 desalting column equilibrated by PBS and 1-5 mM EDTA buffer. Then, the pyridyldithiol group was reduced with 100 mM DTT thus forming thiol-modified protein. The thiolated protein was then separated using a Sephadex G-25 column equilibrated by PBS and 1-5 mM EDTA buffer. Lastly, to prepare antibody-linked liposomes, the thiolated antibodies were immediately mixed with the PEGylated liposome containing maleimide and incubated for 12-18 hours at 4-8° C. To remove non-linked antibodies the solution was then subjected to an ultracentrifuge and spinned at 100,000 g for 1-3 hours. The supernatant was carefully removed. Desired volume of PBS (10 mM Phosphate Buffered Saline)/1-5 M EDTA buffer added to pellets and they were rehydrated during at least 3 hours or overnight at 4-6° C. with gentle stirring. Re-dissolved pellets contain antibody conjugated LUVs and can be used in further tests.

### Method 3.

Conjugation of anti-ASMA antibodies to the prepared PEGylated liposome containing carboxylic acid was based on activation of carboxyl group located on a distal end of PEGylated lipid DSPE-PEG2000 with a mixture [Ethyl-dimethylaminopropyl]carbodiimide hydrochloride (EDC)/Hydroxysulfosuccinimide (NHS). Here EDC is used to activate the carboxyl group on the liposomal surface to create a crosslinker. NHS is added together with EDC to make this linker more stable and avoid undesired hydrolysis. The resulting modified liposome binds to primary amines on the antibody. In contrast to previous methods, activation of carboxyl group on PEGylated liposomes was performed in 0.1M MES buffer, pH6.0. So, unilamellar vesicles were prepared in 0.1M MES buffer and then mixed with EDC/NHS for 15-60 min at room temperature. The molar ratio EDC to NHS varied from 1:1 to 1:4. The molar ratio carboxy-PEG2000-DSPE to EDC varied from 1:5 to 1:20. Non-reacted EDC/NHS were separated from carbodiimide-modified PEGylated liposome by using a Sephadex G-25 column equilibrated by PBS buffer. Lastly, to prepare antibody-linked liposomes, the modified PEGylated liposomes were immediately mixed with corresponding antibodies and incubated for 12-18 hours at 4-8° C. To remove non-linked antibodies the solution was then subjected to an ultracentrifuge and spinned at 100,000 g for 1-3 hours. The supernatant was carefully removed. Desired volume of PBS (10 mM Phosphate Buffered Saline) buffer added to pellets and they were rehydrated during at least 3 hours or overnight at 4-6° C. with gentle stirring. Re-dissolved pellets contain antibody conjugated LUVs and can be used in further tests.

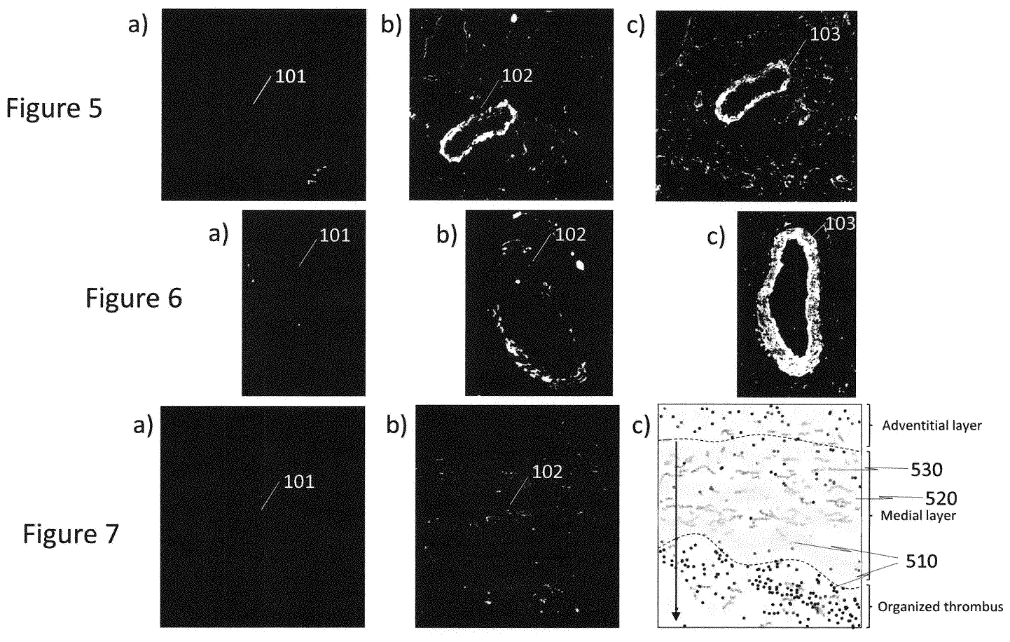

FIGS. 5a-c, FIGS. 6a-c and FIGS. 7a-c show fluorescence stainings for unconjugated targeting liposomes, alpha smooth muscle actin antibody (αSMA-Ab)-conjugated targeting liposomes, and respective control stainings with αSMA-Ab. These liposomes were prepared as described in the foregoing Example 3 with the conjugation method 1.

FIGS. 5a-c show a circular cross-section of an artery in the human tonsil tissue imaged under fluorescence microscope after staining; the artery is negative for the unconjugated targeting liposome, labelled as 101 in FIG. 5a, but positive for the αSMA-Ab-conjugated targeting liposome, labelled as 102 in FIG. 5b and αSMA-Ab, labelled as 103 in FIG. 5c.

FIGS. 6a-c show another example of an artery in the human tonsil tissue; similarly as in the example shown in FIG. 5a-c, the artery is negative for the unconjugated targeting liposome 101 in FIG. 6a, but positive for the αSMA-ab-conjugated targeting liposome 102 in FIG. 6b and αSMA-ab 103 in FIG. 6c.

FIGS. 7a-b show smooth muscle cells in a cross-section of a degenerated human intracranial aneurysm (IA) wall, imaged under fluorescence microscope after staining; the IA wall is negative for the unconjugated targeting liposome 101 in FIG. 7a, but positive for the αSMA-ab-conjugated targeting liposome 102 in FIG. 7b. FIG. 7c shows a schematic drawing of the IA wall cross-section, presented in FIGS. 7a-b. In FIG. 7c reference numeral 510 refers to nucleus, nonspecific to a cell type, 520 refers to αSMA, indicating smooth muscle cells, and 530 refers to a connective tissue, respectively.

Some advantageous embodiments of the liposomes, compounds and methods according to the present disclosure have been described hereinabove. The disclosure is not limited to the aspects and embodiments described above, but the inventive idea can be applied in numerous ways within the scope of the claims.

The features recited in dependent claims are mutually freely combinable unless otherwise explicitly stated.

1-23. (canceled)

24. A targeting method for anchoring a targeting liposome to an intended tissue area secreting at least one vascular inflammatory marker associated with aneurysm, the method comprises
providing a liposome comprising:
at least one type of lipid and an antibody against at least one vascular inflammatory marker associated with aneurysm; and
at least one label and/or a contrast agent, and
administering the liposome to a subject.

25. The targeting method of claim 24, wherein the at least one vascular inflammatory marker comprises α-Smooth muscle cell actin.

26. The targeting method of claim 24, wherein the at least one vascular inflammatory marker is selected from the group consisting of CD31+ Endothelial cell, Cyclo-oxygenase 2, α-Smooth muscle cell actin and CD45+ leukocytes.

27. The targeting method of claim 24, wherein the at least one vascular inflammatory marker is selected from the group consisting of a-smooth muscle cell actin, myosin heavy chain, smoothelin, S100A4, laminin-1, fibronectin, collagens (I, III, IV, V), CD31+ endothelial cell, vascular cell adhesion molecule 1, intercellular adhesion molecule 1, CD34+ pre-endothelial cell, connexins (Cx37, Cx40, Cx43), elastin, neutrophil (CD11b, CD16, and CD66b), CD45+ leukocytes, CD163+ macrophages, CD68+ macrophages, monocyte/macrophage markers (CD4, CD14, CD114, CD11a, CD11b, CD91, CD16), CD3+ T-lymphocytes, lymphocyte markers (CD4, CD8, CD19, CD20, CD24, CD25, CD38, CD22), natural killer cells (CD16, CD56, CD30, CD38), human leukocyte antigen-DR, tryptase/chymase+ mast cells, fibrin, platelets (CD61), plasmin and plasminogen activators, glycophorin A+ red blood cells, serum amyloid A, C-reactive protein, apolipoprotein B-100+ very low density lipoprotein, intermediate density lipoprotein, and low density lipoprotein, apolipoprotein A-1+ high density lipoprotein, apolipoprotein E, ATP-binding cassette transporter, hydroxynonenal+ oxidized lipid, malondialdehyde+ oxidized-lipid, bacteria or bacterial fragments of Porfyromonas gingivalis, Fusobacterium nucleatum, Streptococcus mutans, Agregatibacter actinomycetemcomitans, Treponema denticola, Prevotella intermedia, and/or Tannerella forsythia, lipopolysaccharides, monocyte chemoattractant protein-1, myeloperoxidase, hemeoxygenase 1, prostaglandin E2 receptor, cyclo-oxygenase 2, complement proteins (C5b9, C3a, C5a), interleukins (IL2, IL6, IL1-36), tumor necrosis factor alpha, matrix metalloproteinase 9, matrix metalloproteinase 2, matrix metalloproteinases (1-28), cathepsins (D, G, S, B, K), neutrophil elastase, adipophilin, growth factors (vascular endothelial growth factor, basic fibroblast growth factor, transforming growth factor beta) and their receptors.

28. The targeting method of claim 24, wherein the label and/or contrast agent comprises a moiety encapsulated into the targeting liposome, which moiety is selected from a magnetic moiety, a radioactive moiety, a radionuclide moiety, a luminescent moiety and a fluorescent moiety.

29. The targeting method of claim 24, wherein the at least one vascular inflammatory marker is presented due to an inflammation.

30. The targeting method of claim 29, wherein the inflammation is resulted from an infection.

31. The targeting method of claim 30, wherein the infection is a vasculitis.

32. A targeting liposome comprising:
at least one type of lipid and an antibody against at least one vascular inflammatory marker associated with aneurysm; and
at least one label and/or a contrast agent.

33. The targeting liposome of claim 32, wherein the at least one vascular inflammatory marker comprises a-Smooth muscle cell actin.

34. The targeting liposome of claim 32, wherein the at least one vascular inflammatory marker is selected from the group consisting of CD31+ Endothelial cell, Cyclo-oxygenase 2, α-Smooth muscle cell actin and CD45+ leukocytes.

35. The targeting liposome of claim 32, wherein the at least one vascular inflammatory marker is selected from the group consisting of a-smooth muscle cell actin, myosin heavy chain, smoothelin, S100A4, laminin-1, fibronectin, collagens (I, III, IV, V), CD31+ endothelial cell, vascular cell adhesion molecule 1, intercellular adhesion molecule 1, CD34+ pre-endothelial cell, connexins (Cx37, Cx40, Cx43), elastin, neutrophil (CD11b, CD16, and CD66b), CD45+ leukocytes, CD163+ macrophages, CD68+ macrophages, monocyte/macrophage markers (CD4, CD14, CD114, CD11a, CD11b, CD91, CD16), CD3+ T-lymphocytes, lymphocyte markers (CD4, CD8, CD19, CD20, CD24, CD25, CD38, CD22), natural killer cells (CD16, CD56, CD30, CD38), human leukocyte antigen-DR, tryptase/chymase+ mast cells, fibrin, platelets (CD61), plasmin and plasminogen activators, glycophorin A+ red blood cells, serum amyloid A, C-reactive protein, apolipoprotein B-100+ very low density lipoprotein, intermediate density lipoprotein, and low density lipoprotein, apolipoprotein A-1+ high density lipoprotein, apolipoprotein E, ATP-binding cassette transporter, hydroxynonenal+oxidized lipid, malondialdehyde+ oxidized-lipid, bacteria or bacterial fragments of Porfyromonas gingivalis, Fusobacterium nucleatum, Streptococcus mutans, Agregatibacter actinomycetemcomitans, Treponema denticola, Prevotella intermedia, and/or Tannerella forsythia, lipopolysaccharides, monocyte chemoattractant protein-1, myeloperoxidase, hemeoxygenase 1, prostaglandin E2 receptor, cyclo-oxygenase 2, complement proteins (C5b9, C3a, C5a), interleukins (IL2, IL6, IL1-36), tumor necrosis factor alpha, matrix metalloproteinase 9, matrix metalloproteinase 2, matrix metalloproteinases (1-28), cathepsins (D, G, S, B, K), neutrophil elastase, adipophilin, growth factors (vascular endothelial growth factor, basic fibroblast growth factor, transforming growth factor beta) and their receptors.

36. The targeting liposome of claim 32, wherein the label and/or contrast agent comprises a moiety encapsulated into the targeting liposome, which moiety is selected from a magnetic moiety, a radioactive moiety, a radionuclide moiety, a luminescent moiety and a fluorescent moiety.

37. An imaging method for imaging aneurysm, wherein the imaging method comprises detecting the intended tissue area secreting at least one vascular inflammatory marker associated with aneurysm by using an imaging method that detects the label and/or the contrast agent carried in the targeting liposome of claim 32.

38. The imaging method of claim 37, wherein the imaging method is selected from the group consisting of magnetic resonance imaging (MRI), magnetic resonance angiography (MRA), x-ray imaging, computed tomography (CT), computed tomography angiography (CTA), positron emission tomography (PET), single-photon emission computed tomography (SPECT), and Digital subtraction angiography (DSA).

39. The imaging method of claim 37, wherein the method further comprises selecting a treatment option for the detected aneurysm based on which antibody carried in the targeting liposome of claim 35 is bound to the at least one vascular inflammatory marker secreted from the detected tissue area.

40. The imaging method of claim 39, wherein the imaging comprises a plurality of imaging phases where:
a. in the first imaging phase the intended tissue secreting at least one vascular inflammatory marker associated with aneurysm is detected by using a targeting liposome comprising at least two different antibodies against some vascular inflammatory markers of claim 35, and
b. in at least one subsequent imaging phase the intended tissue secreting at least one vascular inflammatory marker associated with aneurysm is detected by using another targeting liposome that is devoid of at least one antibody compared to the targeting liposome used in the preceding imaging phase.

41. The imaging method of claim 39, wherein the treatment option is selected from the list consisting of clipping, coiling, stenting, and revascularization.

42. The imaging method of claim 37, wherein the at least one vascular inflammatory marker is selected from the group consisting of a-smooth muscle cell actin, myosin heavy chain, smoothelin, S100A4, laminin-1, fibronectin, collagens (I, III, IV, V), CD31+ endothelial cell, vascular cell adhesion molecule 1, intercellular adhesion molecule 1, CD34+ pre-endothelial cell, connexins (Cx37, Cx40, Cx43), elastin, neutrophil (CD11b, CD16, and CD66b), CD45+ leukocytes, CD163+ macrophages, CD68+ macrophages, monocyte/macrophage markers (CD4, CD14, CD114, CD11a, CD11b, CD91, CD16), CD3+ T-lymphocytes, lymphocyte markers (CD4, CD8, CD19, CD20, CD24, CD25, CD38, CD22), natural killer cells (CD16, CD56, CD30, CD38), human leukocyte antigen-DR, tryptase/chymase+ mast cells, fibrin, platelets (CD61), plasmin and plasminogen activators, glycophorin A+ red blood cells, serum amyloid A, C-reactive protein, apolipoprotein B-100+ very low density lipoprotein, intermediate density lipoprotein, and low density lipoprotein, apolipoprotein A-1+ high density lipoprotein, apolipoprotein E, ATP-binding cassette transporter, hydroxynonenal+ oxidized lipid, malondialdehyde+ oxidized-lipid, bacteria or bacterial fragments of Porfyromonas gingivalis, Fusobacterium nucleatum, Streptococcus mutans, Agregatibacter actinomycetemcomitans, Treponema denticola, Prevotella intermedia, and/or Tannerella forsythia, lipopolysaccharides, monocyte chemoattractant protein-1, myeloperoxidase, hemeoxygenase 1, prostaglandin E2 receptor, cyclo-oxygenase 2, complement proteins (C5b9, C3a, C5a), interleukins (IL2, IL6, IL1-36), tumor necrosis factor alpha, matrix metalloproteinase 9, matrix metalloproteinase 2, matrix metalloproteinases (1-28), cathepsins (D, G, S, B, K), neutrophil elastase, adipophilin, growth factors (vascular endothelial growth factor, basic fibroblast growth factor, transforming growth factor beta) and their receptors.

43. The imaging method of claim 37, wherein the label and/or contrast agent comprises a moiety encapsulated into the targeting liposome, which moiety is selected from a magnetic moiety, a radioactive moiety, a radionuclide moiety, a luminescent moiety and a fluorescent moiety.

44. A method for delivering active pharmaceutical ingredient (API) for treating vascular inflammation associated with aneurysm, the vehicle comprising the targeting liposome of claim 32, and the active pharmaceutical ingredient, wherein the targeting liposome is utilised for delivering the API onto the intended tissue area secreting the at least one vascular inflammatory marker associated with aneurysm.

45. The method of claim 44, wherein the at least one vascular inflammatory marker is selected from the group consisting of a-smooth muscle cell actin, myosin heavy chain, smoothelin, S100A4, laminin-1, fibronectin, collagens (I, III, IV, V), CD31+ endothelial cell, vascular cell adhesion molecule 1, intercellular adhesion molecule 1, CD34+ pre-endothelial cell, connexins (Cx37, Cx40, Cx43), elastin, neutrophil (CD11b, CD16, and CD66b), CD45+ leukocytes, CD163+ macrophages, CD68+ macrophages, monocyte/macrophage markers (CD4, CD14, CD114, CD11a, CD11b, CD91, CD16), CD3+ T-lymphocytes, lymphocyte markers (CD4, CD8, CD19, CD20, CD24, CD25, CD38, CD22), natural killer cells (CD16, CD56, CD30, CD38), human leukocyte antigen-DR, tryptase/chymase+mast cells, fibrin, platelets (CD61), plasmin and plasminogen activators, glycophorin A+ red blood cells, serum amyloid A, C-reactive protein, apolipoprotein B-100+ very low density lipoprotein, intermediate density lipoprotein, and low density lipoprotein, apolipoprotein A-1+ high density lipoprotein, apolipoprotein E, ATP-binding cassette transporter, hydroxynonenal+ oxidized lipid, malondialdehyde+ oxidized-lipid, bacteria or bacterial fragments of Porfyromonas gingivalis, Fusobacterium nucleatum, Streptococcus mutans, Agregatibacter actinomycetemcomitans, Treponema denticola, Prevotella intermedia, and/or Tannerella forsythia, lipopolysaccharides, monocyte chemoattractant protein-1, myeloperoxidase, hemeoxygenase 1, prostaglandin E2 receptor, cyclo-oxygenase 2, complement proteins (C5b9, C3a, C5a), interleukins (IL2, IL6, IL1-36), tumor necrosis factor alpha, matrix metalloproteinase 9, matrix metalloproteinase 2, matrix metalloproteinases (1-28), cathepsins (D, G, S, B, K), neutrophil elastase, adipophilin, growth factors (vascular endothelial growth factor, basic fibroblast growth factor, transforming growth factor beta) and their receptors.
